(19) United States
(12) Patent Application Publication
(10) Pub. No.: US 2024/0415440 A1
(43) Pub. Date: Dec. 19, 2024

(54) ELECTROCARDIOGRAM ("ECG") SIGNAL ANALYSIS AND Q-T SEGMENT MEASUREMENT

(71) Applicant: Draeger Medical Systems, Inc., Andover, MA (US)

(72) Inventors: Yu CHEN, Andover, MA (US); Haisheng LU, Andover, MA (US)

(21) Appl. No.: 18/734,992

(22) Filed: Jun. 5, 2024

Related U.S. Application Data
(60) Provisional application No. 63/521,568, filed on Jun. 16, 2023.

(51) Int. Cl. A61B 5/355; A61B 5/00; A61B 5/36

(52) U.S. Cl. CPC A61B 5/355; A61B 5/36; A61B 5/72

(57) ABSTRACT

A method of processing of electrocardiogram (“ECG”) signals from at least one ECG lead connected to a patient includes computing an average beat from a plurality of beats occurring during a predetermined averaging interval. An R-point, an onset point and a J-point of the average beat is computed to establish a Q-T segment of the average beat. The R-point, onset point and the J-point of the average beat are used to determine a Q-T segment for each beat of the averaging interval, and the average of the Q-T segments of each beat is computed and averaged with the Q-T segment of the average beat.

### CROSS-REFERENCE TO RELATED APPLICATION

This application claims priority to U.S. Provisional Patent Application No. 63/521,568 filed Jun. 16, 2023, the contents of which being incorporated herein by reference.

### BACKGROUND

The present disclosure relates generally to the field of electrocardiogram (“ECG”) signal analysis. More particularly, the present disclosure relates to ECG waveform analysis including artifact detection and rejection, and cardiac signal segment measurements.

Electrocardiogram systems are commonly used to monitor patients' heart conditions as well to detect or predict cardiac events and conditions. In clinical settings, ECG signals representative of a patient's condition are captured in waveforms and analyzed by physiological monitoring devices. The physiological monitoring devices identify systolic segments of the captured waveforms, such as QRS-complexes, P-Q segments, S-T segments, and the like. These systolic segments reflect the progression of electrical signals in the heart and corresponding to the depolarization of the right and left ventricles and the contraction of cardiac muscles. For a normal sinus rhythm, an R-wave (a sharp upward deflection) in the QRS-complex with a large amplitude and a small width, is suitable for measuring heart rate and other cardiac conditions. Physiological monitoring devices further classify the detected ECG waveforms into different types, based on features extracted from the morphology of various systolic segment.

In clinical settings, noise contamination caused by artifact signals may adversely impact the precision and accuracy of ECG signal analysis including identification of various systolic segments and subsequent beat classification. For example, noise contamination may impact the accuracy of algorithms designed to detect several cardiac pathologies such as arrhythmias. As a result, a high rate of false arrhythmia alarms may lead to alarm fatigue, where clinicians may be potentially desensitized to frequent invalid or nonactionable alarms and therefore, silencing the alarms with a risk of missing genuine and critical alarms.

A variety of sources may cause artifact signals, including physiological artifacts caused by patients and non-physiological artifacts caused by electric circuitry in the physiological monitoring devices and/or other devices in the clinical environment. Thus, it is important for physiological monitoring devices to accurately detect artifact signals, identify and analyze the corrupted segments of ECG signals contaminated by artifacts.

### SUMMARY

There exists a need for improved detection and analysis of ECG signals, for the physiological monitoring device to identify artifact signals and accordingly, analyze ECG signals in real-time even in the presence of artifacts. There also exists a need for identifying and analyzing systolic segments in real-time in the presence of noise, for heart rate calculation and beat classification, as well as alarm generation.

To resolve or mitigate at least one or more of the above problems and potentially other present or future problems, one aspect of the present disclosure relates to an apparatus for analyzing ECG signals obtained from a patient monitor including one or more ECG leads coupled to the patient.

The apparatus may include one or more processors programmed by machine-readable instructions. The processor(s) may be programmed to select a plurality of sample points from in the plurality of sample signals, extract a plurality of features from the selected plurality of sample points and generate a probability of the existence of the artifact signals in the plurality of sample signals, by applying a transformation process to at least two of the plurality of features. The processor(s) may further be programmed to apply an algorithm to one or more signals to reliably and accurately detect one or more systolic segments of the sensed ECG signals.

One or more examples in the present disclosure provide but are not limited to the following advantages. Note that not all embodiments will necessarily manifest all of these advantages. Furthermore, to the extent that one or more embodiments manifest one or more of the advantages, not all embodiments will manifest such advantages to the same extent or degree.

A variety of artifact signals that cause disturbances in ECG monitoring can be detected in real-time. For example, the embodiments in the present disclosure are capable of detecting physiological artifacts caused by patient motion, including motions associated with the patient's medical conditions (e.g., tremors, shivering) and regular muscular activities (e.g., brushing, combing). Additionally, the embodiments in the present disclosure are capable of detecting non-physiological artifacts including electromagnetic interference caused by physiological monitoring systems or other electrical devices in the clinical environment, as well as artifacts caused by cable and/or electrode malfunction. Note that not all embodiments will necessarily exhibit all these advantages nor will they exhibit them to the same degree. Thus, the embodiments in the present disclosure prevent artifact signals from being falsely identified as part of a QRS-complex, thereby increasing the accuracy in QRS-complex identification and classification, as well as the accuracy in heart rate calculation and alarm generation.

On the other hand, when a patient has certain medical conditions, the morphologies of the monitored ECG waveforms can be complex or irregular, and thus, difficult to differentiate from artifact signals. The examples in the present disclosure provide validation processes for identified artifact signals, thereby reducing the false-positive rate. With the complex or irregular ECG waveforms being accurately identified rather than treated as artifacts, the examples in the present disclosure are capable of analyzing different types of ECG waveforms accurately and generating alarms. Thus, clinical providers can promptly identify the medical conditions of the patient and provide treatment as needed, thereby improving clinical workflows.

The above presents a simplified summary in order to provide a basic understanding of some aspects of what is claimed below. This summary is not an exhaustive overview of the claimed subject matter. It is not intended to identify key or critical elements of the disclosure or to delineate the scope of the claims. Its sole purpose is to present some concepts in a simplified form as a prelude to the more detailed description that is discussed later.

### BRIEF DESCRIPTION OF THE DRAWINGS

In the drawings, like reference numbers generally indicate identical, functionally similar, and/or structurally similar elements.

FIG. 1 illustrates a patient monitoring system according to one or more examples;

FIG. 2 is a schematic representation of a normal sinus rhythm ECG wave illustrating selected characteristics thereof;

FIGS. 3A and 3B together comprise a flow diagram illustrating a method of processing ECG signals according to one or more examples;

FIG. 4 is a flow diagram illustrating a method of Q-T segment measurement according to one or more examples;

FIG. 5A illustrates a pair of sensed ECG signals before processing according to an ECG processing algorithm according to one or more examples;

FIG. 5B illustrates a pair of sensed ECG signals after processing according to an ECG processing algorithm according to one or more examples;

FIGS. 6A-6D illustrate distinct morphologies of T-waves end search windows based thereon;

FIG. 7 is a flow diagram illustrating a method of Q-T segment measurement according to one or more examples;

FIG. 8 is a block diagram of a computing resource implementing a method of operating a patient monitoring system according to one or more examples; and

FIG. 9 is a block diagram of a computing resource implementing a method of operating a patient monitoring system according to one or more examples.

While the disclosed subject matter is susceptible to various modifications and alternative forms, the drawings illustrate specific implementations described in detail by way of example. It should be understood, however, that the description herein of specific examples is not intended to limit that which is claimed to the particular forms disclosed, but on the contrary, the intention is to cover all modifications, equivalents, and alternatives falling within the spirit and scope of the appended claims.

### DETAILED DESCRIPTION

Illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

As used herein, to “provide” an item means to have possession of and/or control over the item. This may include, for example, forming (or assembling) some or all of the item from its constituent materials and/or, obtaining possession of and/or control over an already-formed item.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted. In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

ECG signal processing, as used herein, refers to, without limitation manipulating an analog signal in such a way that the signal meets the requirements of a next stage for further processing. ECG signal processing may include converting between analog and digital realms (e.g., via an analog-to-digital or digital-to-analog converter), amplification, filtering, converting, biasing, range matching, isolation and any other processes required to make a sensor output suitable for processing.

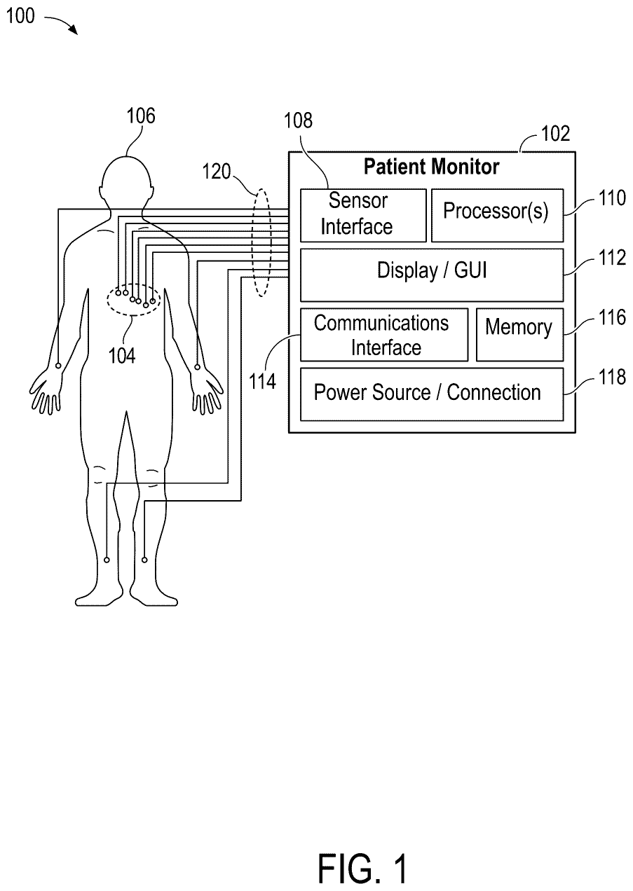

Turning now to the drawings, FIG. 1 shows a physiological monitoring system 100 according to one or more examples. As shown in FIG. 1, the system 100 includes a patient monitor 102 (e.g., a physiological monitoring device) capable of receiving physiological data from various sensors 104 connected to a patient 106. In this example, sensors 104 comprise ECG electrodes affixed to the skin of patient 106.

In general, it is contemplated by the present disclosure that patient monitor 102 includes electronic components and/or electronic computing devices operable to receive, transmit, process, store, and/or manage patient data and information associated performing the functions of the system as described herein, which encompasses any suitable processing device adapted to perform computing tasks consistent with the execution of computer-readable instructions stored in a memory or a computer-readable recording medium.

Further, any, all, or some of the computing devices in patient monitor 102 may be adapted to execute any operating system, including Linux®, UNIX®, Windows Server®, etc., as well as virtual machines adapted to virtualize execution of a particular operating system, including customized and proprietary operating systems. Patient monitor 102 may be further equipped with components to facilitate communication with other computing devices over one or more network connections, which may include connections to local and wide area networks, wireless and wired networks, public and private networks, and any other communication network enabling communication in the system.

As shown in FIG. 1, patient monitor 102 may be, for example, a patient monitor implemented to monitor various physiological parameters of patient 106 via sensors 104. Patient monitor 102 may include a sensor interface 108, one or more processors 110, a display/graphical user interface (“GUI”) 112, a communications interface 114, a memory 116, and a power source (or power connection) 118. Sensor interface 108 may be implemented in hardware or combination of hardware and software and is used to connect via wired and/or wireless connections to sensors 104 for gathering physiological data from the patient 106. As noted, sensors 104 in the present example are ECG electrodes affixed to the skin of patient 106. A plurality of conductive leads 120, comprising a plurality of conductive cables, are provided for coupling sensors 104 to sensor interface 108. In one or more examples, conductive leads 120 comprise a plurality of ECG cables.

The data signals from the sensors 104 may include, for example, sensor data related to an ECG. The one or more processors 110 may be used for controlling the general operations of patient monitor 102, as well as processing sensor data received by sensor interface 108. Such circuitry for processing sensor data is variously referred to herein as “signal processing circuitry” and “signal processors.” Such elements may include programmed processors and associated memory, dedicated processing circuits, or a combination thereof. The one or more processors 110 may be, but are not limited to, a central processing unit (“CPU”), a hardware microprocessor, a multi-core processor, a single core processor, a field programmable gate array (“FPGA”), a microcontroller, an application specific integrated circuit (“ASIC”), a digital signal processor (“DSP”), or other similar processing device capable of executing any type of instructions, algorithms, or software for controlling the operation and performing the functions of patient monitor 102. In some embodiments, the one or more processors 110 may comprise a processor chipset including, for example and without limitation, one or more co-processors.

Display/GUI 112 may be configured to display various patient data, sensor data, and hospital or patient care information, and includes a user interface implemented for allowing interaction and communication between a user and patient monitor 102. Display/GUI 112 may include a keyboard (not shown) and/or pointing or tracking device (not shown), as well as a display, such as a liquid crystal display (“LCD”), cathode ray tube (“CRT”) display, thin film transistor (“TFT”) display, light-emitting diode (“LED”) display, high definition (“HD”) display, or other similar display device that may include touch screen capabilities. Display/GUI 112 may provide a means for inputting instructions or information directly to the patient monitor 102. The patient information displayed may, for example, relate to the measured physiological parameters of patient 106 (e.g., ECG readings).

Communications interface 114 may enable patient monitor 102 to directly or indirectly (via, for example, a monitor mount) communicate with one or more computing networks and devices, workstations, consoles, computers, monitoring equipment, alert systems, and/or mobile devices (e.g., a mobile phone, tablet, or other hand-held display device). Communications interface 114 may include various network cards, interfaces, communication channels, cloud, antennas, and/or circuitry to enable wired and wireless communications with such computing networks and devices. Communications interface 114 may be used to implement, for example, a Bluetooth® connection, a cellular network connection, and/or a WiFi® connection with such computing networks and devices. Example wireless communication connections implemented using the communication interface 6 include wireless connections that operate in accordance with, but are not limited to, IEEE802.11 protocol, a Radio Frequency For Consumer Electronics (“RF4CE”) protocol, and/or IEEE802.15.4 protocol (e.g., ZigBee® protocol). In essence, any wireless communication protocol may be used.

Additionally, communications interface 114 may enable direct (i.e., device-to-device) communications (e.g., messaging, signal exchange, etc.) such as from a monitor mount to patient monitor 102 using, for example, a universal serial bus (“USB”) connection or other communication protocol interface. The communication interface 6 may also enable direct device-to-device connection to other devices such as to a tablet, computer, or similar electronic device; or to an external storage device or memory.

Memory 116 may be a single memory device or one or more memory devices at one or more memory locations that may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. Memory 116 may be used to store any type of instructions and patient data associated with algorithms, processes, or operations for controlling the general functions and operations of patient monitor 102.

Power source 118 may include a self-contained power source such as a battery pack and/or include an interface to be powered through an electrical outlet (either directly or by way of a monitor mount). Power source 118 may also be a rechargeable battery that can be detached allowing for replacement. In the case of a rechargeable battery, a small built-in back-up battery (or super capacitor) can be provided for continuous power to be provided to patient monitor 102 during battery replacement. Communication between the components of patient monitor 102 in this example (may be established using an internal bus (not explicitly shown in FIG. 1).

Patient monitor 102 may be attached to one or more of several different types of sensors 104 and may be configured to measure and readout physiological data related to patient 106. As noted, sensors 104 may be attached to patient monitor 102 by conductive leads 120 which may be, for example, cables coupled to sensor interface 108. Additionally, or alternatively, one or more sensors 104 may connected to sensor interface 108 via a wireless connection. In which case sensor interface 108 may include circuitry for receiving data from and sending data to one or more devices using, for example, a WiFi® connection, a cellular network connection, and/or a Bluetooth® connection.

The data signals received from sensors 104, may be analog signals. For example, the data signals for the ECG may be input to sensor interface 108, which can include an ECG data acquisition circuit (not shown separately in FIG. 1). An ECG data acquisition circuit may include amplifying and filtering circuitry as well as analog-to-digital (A/D) circuitry that converts the analog signal to a digital signal using amplification, filtering, and A/D conversion methods. In the event that the ECG sensor is a wireless sensor, sensor interface 108 may receive the data signals from a wireless communication module. Thus, sensor interface 108 is a component which may be configured to interface with one or more sensors 104 and receive sensor data therefrom.

As further described herein, the processing performed by an ECG data acquisition circuit may generate analog data waveforms or digital data waveforms that are analyzed by, in this particular embodiment, a microcontroller. However, other embodiments may use other kinds of processors. The microcontroller may be one of the processors 110.

The one or more processors 110, for example, may analyze the ECG waveforms to identify certain waveform characteristics and threshold levels indicative of conditions (abnormal and normal) of the patient 106 using one or more monitoring methods. A monitoring method may include comparing an analog or a digital waveform characteristic or an analog or digital value to one or more threshold values and generating a comparison result based thereon. The microcontroller may be, for example, a processor, an FPGA, an ASIC, a DSP, a microcontroller, or similar processing device. The microcontroller may include a memory or use a separate memory 116. The memory may be, for example, a RAM, a memory buffer, a hard drive, a database, an EPROM, an EEPROM, a ROM, a flash memory, a hard disk, or any other non-transitory computer readable medium.

Memory 116 may store software or algorithms with executable instructions and the microcontroller may execute a set of instructions of the software or algorithms in association with executing different operations and functions of patient monitor 102 such as analyzing the digital data waveforms related to the data signals from sensors 104.

As noted, in the example of FIG. 1, conductive leads 120 between sensors 104 and sensor interface 108 may be an ECG lead set. Conductive leads 120 typically terminate at a sensor 104 that is attached to the patient for measuring ECG data.

As noted, an ECG signal reflects electrical impulses in the heart associated with the systolic rhythm of a beating heart. A normal sinus rhythm includes a repeating succession of “heartbeats” corresponding to heart contractions, and an ECG signal may reflect various phases of such contractions.

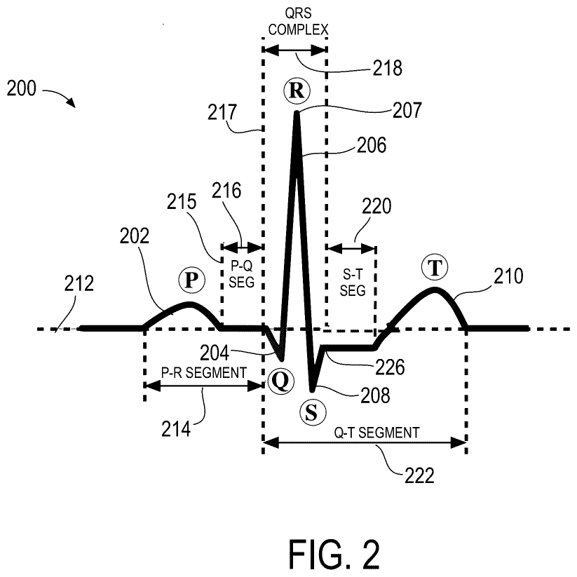

FIG. 2 is a schematic representation of a “normal” sinus rhythm ECG wave 200. illustrates a sensed ECG signal corresponding to various phases of a single cardiac beat. As shown in FIG. 2, ECG wave 200 includes a plurality of distinct phases corresponding to periods of polarization and depolarization of regions of the cardiac muscle. In particular, ECG wave 200 includes a P-wave 202, a Q-wave 204, an R-wave 206 having a peak at a fiducial point 207 (also referred to herein as an “R-point”), an S-wave 208, and a T-wave 210. Each of these waves represents either a positive or negative polarization relative to an ECG baseline 212.

A normal sinus rhythm ECG wave such as ECG wave 200 is commonly characterized according to a number of segments, including, as shown in FIG. 2, a P-R segment 214, a P-Q segment 216, a QRS complex 218, an S-T segment 220, and a Q-T segment 222. A normal sinus rhythm ECG wave such as ECG wave 200 may further be characterized by an “isoelectric point” (occurring at dashed line 215 in FIG. 2), an “onset” (occurring at dashed line 217 in FIG. 2), and a junction point or “J-point” 226. The J-point is the junction between the termination of the QRS complex 218 and the onset of the S-T segment 220. Referring to FIG. 2, J-point 226 in ECG wave 200 is the point where QRS complex 218 joins the S-T segment 220. J-point 226 represents the approximate end of depolarization and the beginning of repolarization. J-point 226 may deviate from baseline 212.

As noted above, noise and artifacts may be introduced into sensed ECG signals, such as baseline wander caused by motion of the patient or the leads, muscle/electromyographc (“EMG”) artifacts, spectrum overlapping with the ECG signal, electrode motion artifacts, respiration artifacts, lead placement artifacts, and so on, which can cause the baseline (such as baseline 212 in FIG. 2) to vary. Variability of the ECG baseline can make it difficult to clinically assess a patient's ECG reading and reliably identify the various waves and segments, in the sensed ECG waveform.

Referring again to FIG. 1, the one or more processors 110 may further execute under programmed control processes for performing systolic segment measurements, such as S-T segment measurements, Q-T segment measurements, QRS-complex detection, and QRS-complex feature extraction. A QRS-complex is commonly the central and most visually obvious part of an ECG waveform, with a duration of approximately 80 milliseconds (mSec)-100 mSec in adults. Patient monitor 102 may identify the QRS-complex by, for example, identifying an R-wave within the QRS-complex. Processor(s) 110 may search one or more edge points of received sample signals, including a starting point, a peak point, a tail point, and an endpoint. By defining one or more edge points, processor(s) 110 may identify the R-wave and its corresponding QRS-complex.

Concurrently or subsequently, patient monitor 102 may further extract one or more features from the identified QRS-complexes, including but not limited to amplitude, width, morphology, curvature, symmetry, peak direction, segments of different waves including R-R segments (i.e., the time interval between two consecutive R-waves), P-R segments (time interval between the beginning of the upslope of the P wave to the beginning of QRS wave), S-T segment measurements, and Q-T segment measurements. Based on these extracted features, patient monitor 102 may further classify the QRS-complexes into different types referred to as “beats”, including a normal beat or a bundle branch block beat (N), a ventricular ectopic beat (V), a supraventricular ectopic beat(S), a fusion of ventricular and normal beat (F) and a paced beat or a beat that cannot be classified (Q). Each beat type has its characteristic features and accordingly, physiological monitoring device 102 may store ECG template databases including various pre-determined threshold values or ranges of pre-determined threshold values for each feature. When a new QRS-complex is identified, patient monitor may extract one or more features and compare them with pre-determined threshold values or a ranges of threshold values, thereby classifying the QRS-complex into a specific beat type based on the comparison results.

In other examples, pre-determined threshold values or the ranges of threshold values may be dynamically updated. That is, after a new beat is classified, the extracted features of this beat are used to update the existing QRS-complex template database. When the identified QRS-complex and its corresponding classification indicate cardiac conditions, processor(s) 110 may generate alarms and display one or more extracted features for clinical providers. For example, the duration, amplitude, and morphology of the QRS-complex are useful for clinical providers to diagnose cardiac arrhythmias, conduction abnormalities, ventricular hypertrophy, myocardial infarction, electrolyte derangements, and other cardiac conditions.

In clinical settings, artifact signals are often mixed with ECG signals, which makes it a challenge to systolic event identification, feature extraction, and subsequently, beat classification. For example, an artifact signal with a high amplitude and narrow width (e.g., high-frequency artifacts and low-frequency artifacts) may adversely impact the identification of S-T segments, R-waves and subsequent feature extraction (e.g., R-R segment, P-R segment, R-wave amplitude, S-T segment, and Q-T segment). On the other hand, baseline shift artifacts may corrupt the ECG signals and interfere with the identification and measurement of S-T segments and Q-T segments, which are important diagnostic markers for various cardiac conditions.

The Q-T segment of an ECG waveform represents the duration of ventricular depolarization and subsequent repolarization, i.e., from the onset point though the end of the T-wave. The Q-T segment may be used clinically as an indirect measure of the repolarization time. Acute increases in the Q-T segment may be observed in multiple clinical situations and may be associated with an increased risk of syncope and sudden death from ventricular arrhythmias. The Q-T segment may be monitored periodically for possible prolongation. Thus, in some situations, it may be desirable to accurately and continuously monitor the Q-T segment in real time in a reliable manner, and to accurately detect feature points of the onset point and the T-wave end in ECG signals.

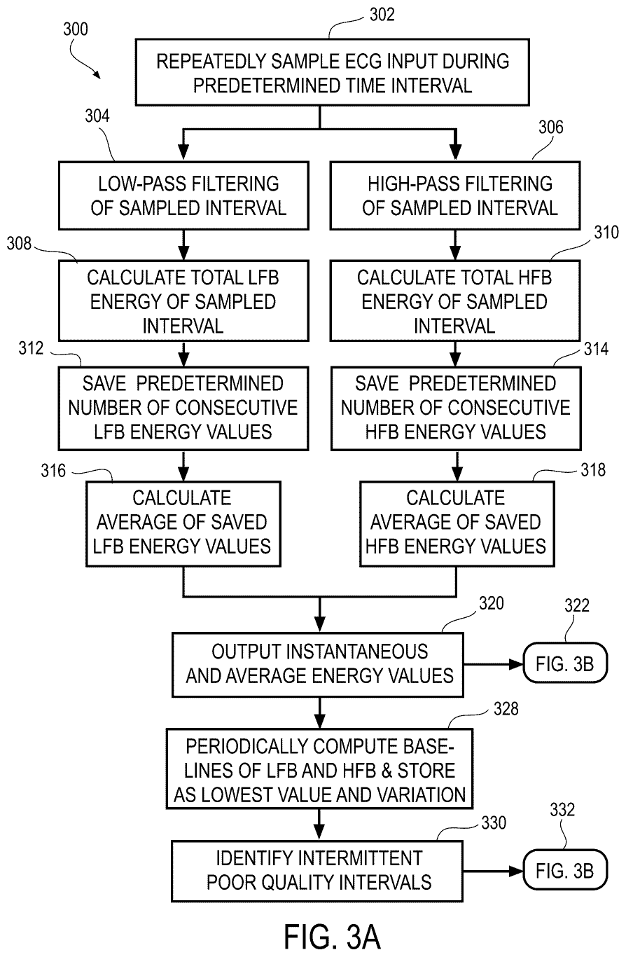

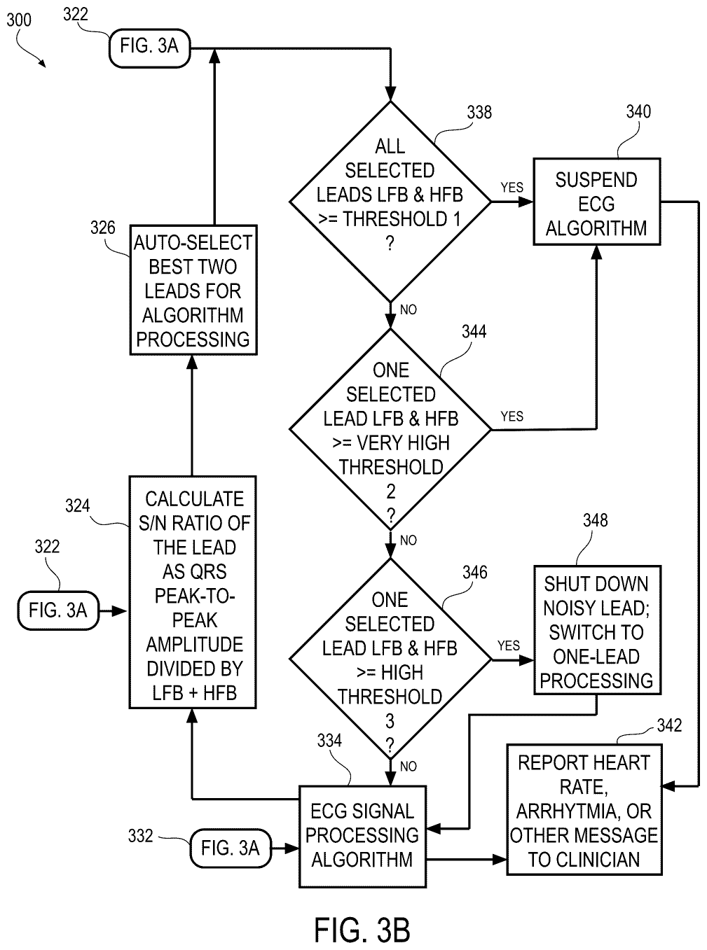

FIGS. 3A and 3B illustrate a method 300 for performing ECG signal processing according to one or more examples. Method 300 may be performed by patient monitor 102, and may be performed independently for each of multiple ECG leads associated with physiological monitoring system 100. The steps of method 300 depicted in FIGS. 3A and 3B may be performed in the digital realm, the analog realm, or a combination thereof.

As shown in FIG. 3A, method 300 begins at block 302 repeated sampling of the ECG signal on each ECG lead over a predetermined time interval. In examples, the ECG signal may be sampled 250 times per second over a 300 mSec interval, although different sampling rates and interval durations may be specified. The acquired sampled waveform is subjected to low-pass filtering in block 304 and to high-pass filtering in block 306. The low-pass filtering (block 304) and the high-pass filtering (block 306) may be performed concurrently or sequentially depending on the embodiment. Furthermore, in each of the low-pass filtering (block 304) and the high-pass filtering (block 306), the signals may be filtered concurrently or sequentially depending on the embodiment. Either finite impulse response (“FIR”) or infinite impulse response (“IIR”) filters may be utilized. In examples, low-pass filtering may comprise filtering with a bandpass window of less and 1 Hz, and high-pass filtering may comprise filtering with a bandpass window higher than 20 Hz.

Thereafter, the total low-frequency bandpass (“LFB”) energy is calculated in block 308, and the total high-frequency bandpass (“HFP”) energy is calculated in block 310. The process of sampling the ECG input (block 302), low-pass filtering (block 304), and calculation of LFG and HFB energies (blocks 308 and 310, respectively) may be repeated continuously, such that, in block 312, a plurality of consecutive LFB energy values are stored in an array. For example, eight consecutive values, corresponding to 2.4 seconds of data, may be stored in block 312. Correspondingly, a plurality of consecutive HFB energy values are stored, in block 314.

In block 316, an average of the plurality of LFB energy values saved in block 312 is calculated, while in block 318, an average of the HFB energy values saved in block 314 is calculated. In block 320, the instantaneous LFB energy values and HFB energy values (from blocks 308 and 310, respectively), and the average LFB and HFB energy values (from blocks 316 and 318, respectively) are outputted. As shown by connecting block 322 in FIGS. 3A and 3B, these outputted values are used in block 324 to calculate a signal-to-noise (“S/N”) ratio of the lead, by dividing the QRS peak-to-peak amplitude value divided by the sum of the LFB and HFB energy values.

Assuming that method 300 of FIGS. 3A and 3B is being performed independently on multiple leads, then as shown in FIG. 3B, in block 326 a selection is made of the best two leads to be utilized for further ECG processing according to the algorithm described in various examples herein. This selection in block 326 may be made based upon the signals having optimal S/N ratios, for example, S/N ratios greater than 10-12 db. as computed in block 324, for example. That is, in the illustrated example the “best” two leads will be those having the greatest S/N ratios. Other embodiments may use other measures for what is “best”.

Referring again to FIG. 3A, method 300 may further involve, in block 328, periodically computing energy baselines of the LFB and HFB energies. These baselines may be computed, for example, in terms of a lowest value and a variation value, i.e., a range of values, for each of the LFB and HFB energy values. The computation step of block 328 may be performed, for example, at 300 mSec intervals based on the past 10-to 20-seconds of averaged data from blocks 316 and 318.

In block 330, intermittent periods of poor ECG signal quality may be identified by comparing the LFB and HFB energy baselines computed in block 328. As shown by connecting block 332 in FIGS. 3A and 3B, this information regarding intermittent poor-quality intervals is provided to a block 334 for performing an ECG signal processing algorithm.

With continued reference to FIG. 3B, execution of the ECG processing algorithm according to one or more examples herein is based upon a selection of leads to be used for that purpose. The selection of leads begins with calculation of S/N ratios for each lead, in block 324 as described above. In examples, the intermittent periods of poor ECG signal quality may be excluded from consideration by the ECG processing algorithm performed in block 334. The ECG algorithm is performed on the beats sensed on the ECG leads selected in block 326. Connecting block 322 in FIG. 3B reflects that the instantaneous and averaged energy values outputted from block 320 in FIG. 3A may be utilized in the assessment of ECG signals provided to the ECG signal processing algorithm (block 334) through a series of threshold assessments (blocks 338, 344, and 346) as herein described.

In particular, as the ECG signal processing algorithm is being performed in block 334, determinations are made, in decision block 338, whether all of the selected leads have LFB and HFB energies which are greater than or equal to a first predetermined threshold. If that is the case, then in block 340, the ECG algorithm is suspended, as this reflects a scenario where the ECG quality is insufficient to be relied upon for making clinical decisions. Upon suspension of the ECG algorithm in block 340, method 300 proceeds to block 342, in which the clinician may be advised of the patient's heart rate, an arrhythmia, S-T segment, or other message.

If all selected leads do not have LFB and HFB energies which exceed the first threshold in block 338, then a further determination is made, in block 344, whether at least one of the selected leads has LFB and HFB energy which exceeds a very high second predetermined threshold, in which case operation proceeds again to block 340, in which the ECG processing algorithm is suspended.

If neither selected lead has LFB and HFB energy exceeding the very high second threshold in decision block 344, then in decision block 346, a determination is made whether one of the selected lead has LFB and HFB energy exceeding a high third predetermined threshold. If not, the normal algorithm operation continues, beginning again at block 334. If one of the selected leads does have LFB and HFB energy exceeding the third high predetermined threshold (which may be lower than the very high first predetermined threshold, this signifies that the lead generating the signal is unsuitably noisy. In that case, in block 348, the noisy lead is shut down and one-lead operation is commenced. A report may be made from block 342 advising the clinician that one-lead operation has commenced.

In some examples, an ECG signal processing algorithm such as represented by block 324 in FIG. 3B may, among multiple processes, perform Q-T segment measurements based upon the lead(s) selected as described herein with reference to FIGS. 3A and 3B. These measurements, or alerts based upon them, may be reported to the clinician in block 342 as shown in FIG. 3B. There are several challenges associated with accurate S-T segment measurement, including strong respiration artifacts in the sensed ECG signals, and baseline noise in the sensed ECG signals.

In cases of respiration artifacts in the sensed ECG signals, it is necessary to eliminate the artifact while preserving the low-frequency signal content especially important for S-T segment measurement, such as prescribed by industry standards such as promulgated by the Association for the Advancement of Medical Instrumentation (AAMI) and the International Electrotechnical Commission (IEC), particularly, IEC/AAMI Standard 60601 2-25.

The baseline noise including any respiration artifacts may be low-frequency signals (e.g., below 0.8-1.0 Hz). The frequency in an ECG signal is typically above 0.05 Hz. Thus, the frequency band of the baseline noise may overlap with the ECG signal of interest, such that a traditional finite impulse response (FIR) or infinite impulse response (IIF) high-pass filter with 0.05 Hz can meet the standard, but may not be sufficient to remove the baseline noise.

Accordingly, in some examples, filtering methodologies may be employed to remove baseline noise. One such methodology is disclosed in U.S. Pat. No. 9,480,411 to Zheng et al., entitled “Electrocardiogram Baseline Removal” (“the '411 patent”) involves estimation of a baseline variation of the ECG signal from a filtered ECG signal and subtracting the estimated baseline variation from the original ECG to produce a baseline-removed ECG signal. The Zheng et al. '411 patent is hereby incorporated by reference herein in its entirety.

Furthermore, some professional recommendations, such as in the American Association of Critical Care Nurses (AACN) “Practice Alert of Ensuring Accurate ST-Segment Monitoring” suggest that clinicians perform further assessment (beyond baseline removal) in response to an S-T segment alarm indicating an S-T segment deviation lasting more than a full minute.

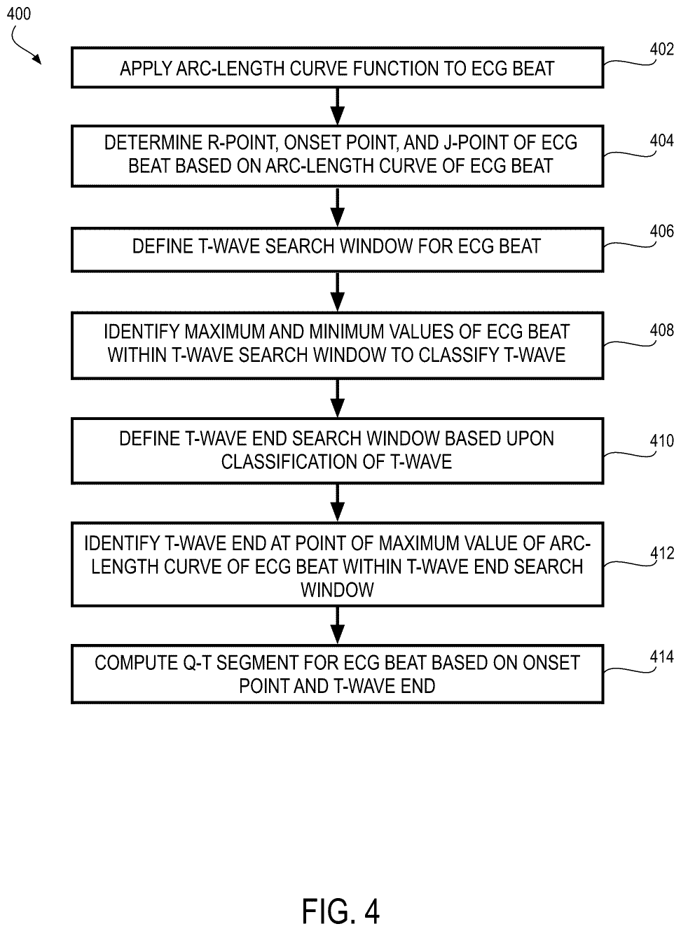

FIG. 4 is a flow diagram illustrating a method 400 for Q-T segment measurement according to one or more examples. It is to be understood that the various steps depicted in FIG. 4 may be performed in a sequence other than that shown in FIG. 4. For example, some steps may be performed on an ongoing basis at the same time as other are being performed.

As shown in FIG. 4, a first step 402 is to apply an arc-length curve function to an ECG signal comprising a plurality of ECG beats. In particular, the ECG signal is subjected to an arc length curve computation according to the following Equation (1):

$$ArcL [ i ] = {\sum }_{k = i - w}^{i} \sqrt{C + {( Δ y )}_{k}^{2}} \\ Δ {y}_{k} = ecgwaveform [ k ] - ecgwaveform [ k - 1 ] \qquad Eq . ( 1 )$$

where i is the time index, C is a constant related to sample interval in mSec, w is the duration of the time window and should be approximately equal to the width of the widest QRS complex.

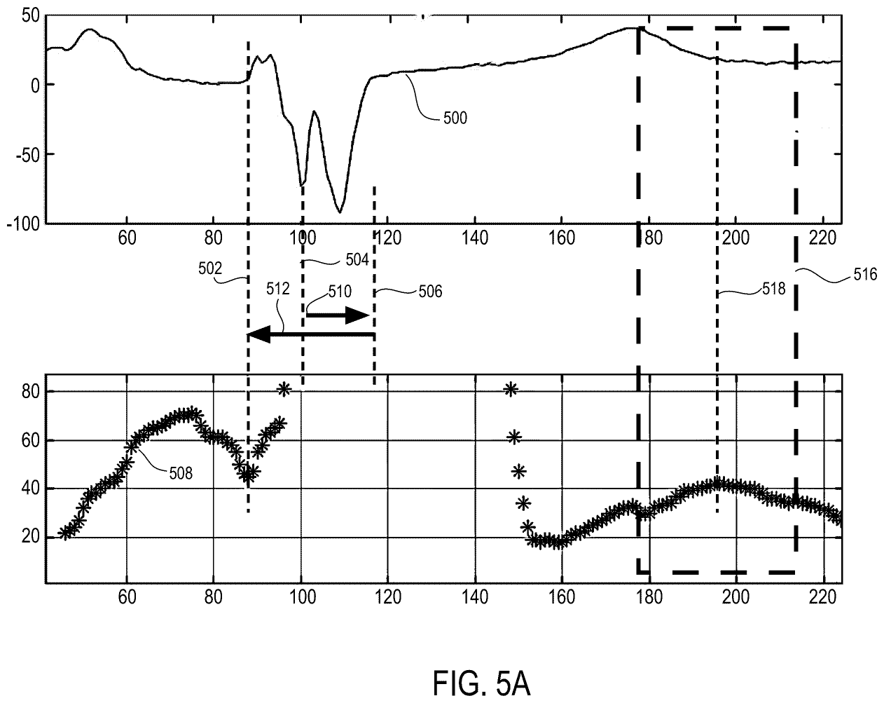

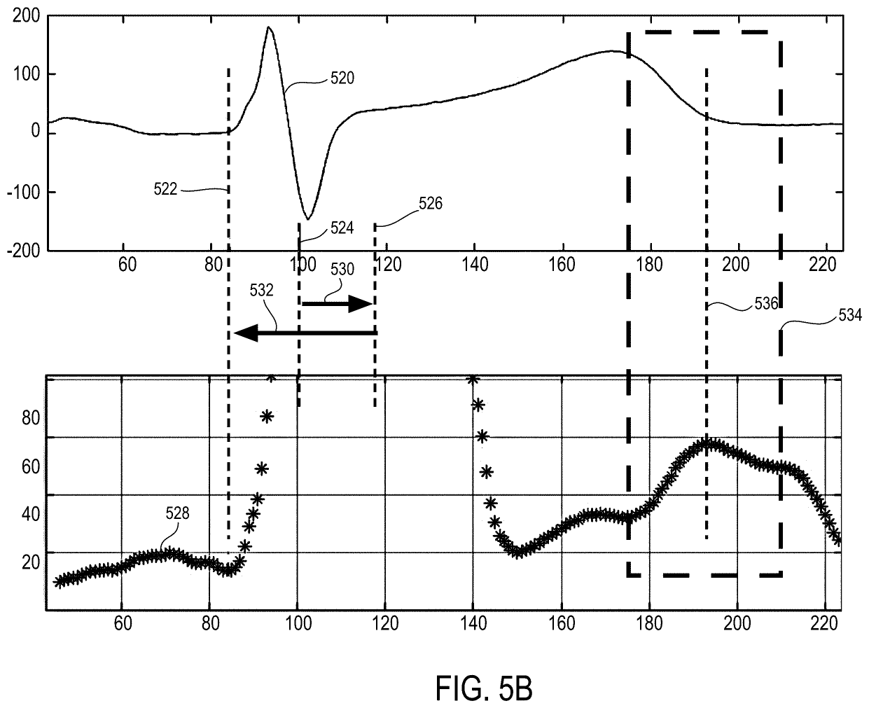

FIGS. 5A and 5B illustrate how the arc-length curve algorithm reflected in Equation (1) above may be used to detect onset points and J-points on two independent leads. In FIG. 5A, an ECG signal 500 from a first lead is shown. Dashed line 502 represents the onset point of ECG signal 500. Dashed line 504 represents the R-point of averaged ECG signal 500. Dashed line 506 identifies the J-point of ECG signal 500.

Similarly, in FIG. 5B, an ECG signal 520 from a second lead is shown. Dashed line 522 represents the onset point of ECG signal 520. Dashed line 524 represents the R-point of ECG signal 520. Dashed line 526 identifies the J-point of ECG signal 520.

Referring again to FIG. 4, computations according to Eq. (1) result in an arc-length curve 508 corresponding to ECG signal 500 shown in FIG. 5A. On arc-length curve 508, from the known R-point (dashed line 504), in block 404, the algorithm searches forward, as indicated by arrow 510 to identify the first point with a maximum value to be identified as the J-point (dashed line 506 in FIG. 5A). Next, as represented by arrow 512 in FIG. 5A, the algorithm searches back from the J-point a predetermined period of time (e.g., the R-J interval plus 60 mSec) to identify the first point with a minimum value to be identified as the onset point (dashed line 502 in FIG. 5A).

Similarly, computations according to Eq. (1) result in an arc-length curve 528 corresponding to ECG signal 520 shown in FIG. 5B. On arc-length curve 528, from the known R-point (dashed line 524), in block 404, the algorithm searches forward, as indicated by arrow 530 to identify the first point with a maximum value to be identified as the J-point (dashed line 526 in FIG. 5B), Next, as represented by arrow 532 in FIG. 5B, the algorithm searches back from the J-point a predetermined period of time (e.g., the R-J interval plus 60 mSec) to identify the first point with a minimum value to be identified as the onset point (dashed line 522 in FIG. 5B).

Next, in step 406, a T-wave search window is defined for each ECG beat. In examples, the beginning of a T-wave search window of an ECG signal begins at the J-point plus a predetermined interval, e.g., 40 mSec, and an ending of the T-wave search window is the lesser of 0.7 times the R-R interval of the ECG signal (0.7×RR) and 800 mSec) after the fiducial point (reference numeral 207 in FIG. 2). That is (using the 40 mSec predetermined interval example noted above):

$$J - pt . + 40 mSec < T - Wave Search Window < Fiducial Pt + min \{ 0.7 \times RR , 800 mSec \}$$

Clinically, T-waves in ECG signals are classified as (i) positive, (ii) negative (inverted), (iii) biphasic/negative, and (iv) biphasic/positive.

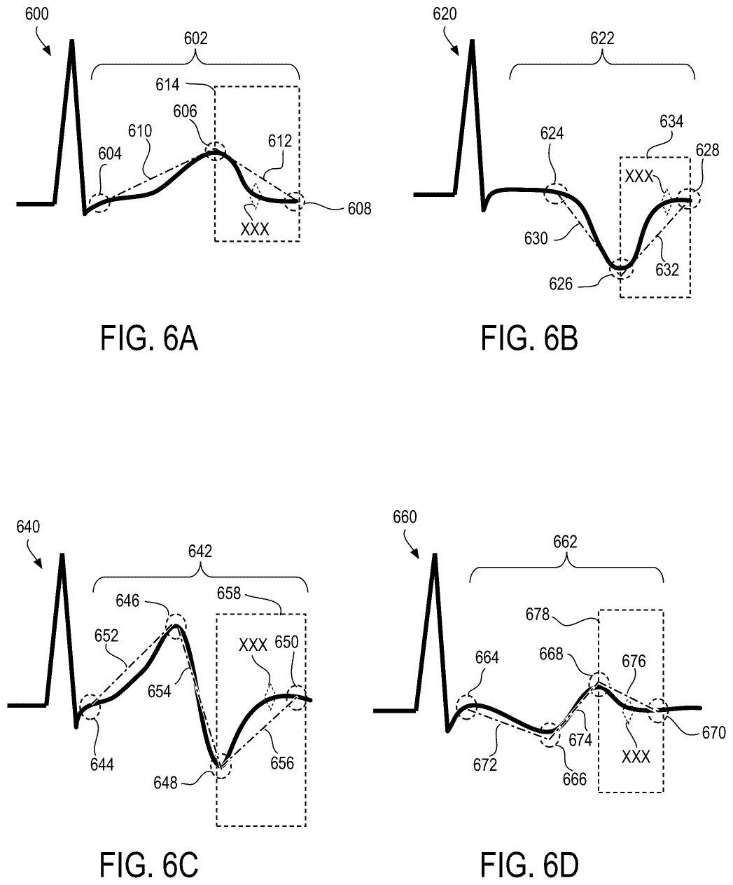

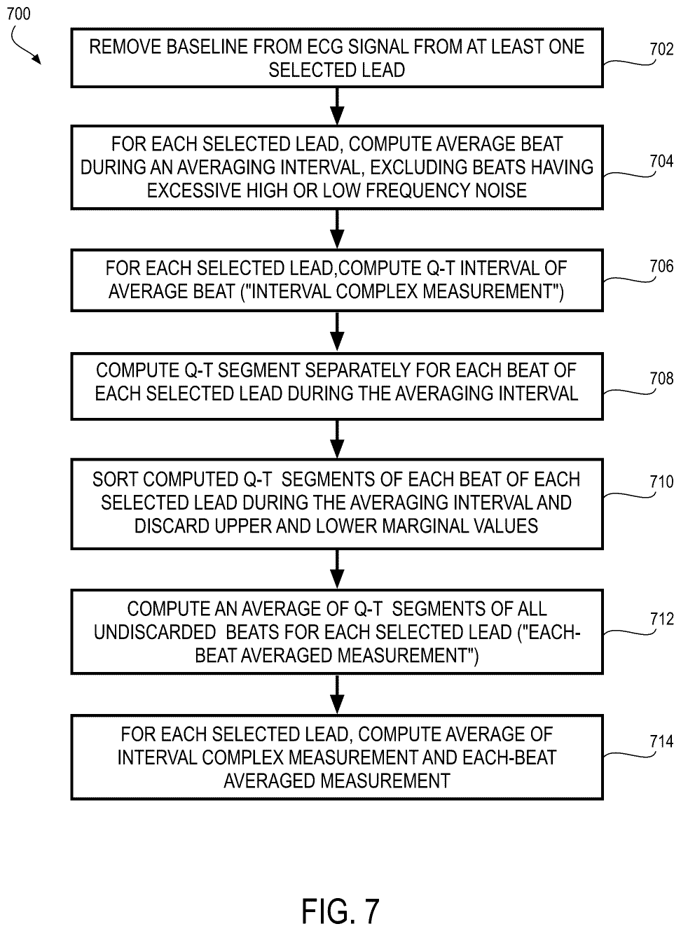

FIGS. 6A-6D illustrate examples of the different classifications of T-waves. In particular, FIG. 6A illustrates an ECG beat 600 having a positive T-wave, designated generally with reference numeral 602. FIG. 6B illustrates an ECG beat 620 having a negative (inverted) T-wave 622. FIG. 6C illustrates an ECG beat 640 having a biphasic/negative T-wave 644. FIG. 7D illustrates an ECG beat 660 having a biphasic/positive T-wave 662.

In order to classify the T-wave of an ECG signal, in block 408, in some examples, the relative maximum and minimum values of the ECG signal in the T-wave search window are identified. The terms “relative maximum” and “relative minimum” are intended to reflect the avoidance of identifying local maximum and minimum values. In examples, the magnitude of a relative maximum value must be greater than all points within a specified window around it, and the magnitude of a relative minimum value must be less than all points within the specified window, and there is preferably a clear change in polarity from one minimum to the next maximum and vice versa. The length of the specified window depends upon the frequency range of the signal of interest.

In the positive T-wave example of FIG. 6A, a first relative minimum is identified with reference numeral 604, a first relative maximum is identified with reference numeral 606, and a second relative minimum is identified with reference numeral 608.

In the negative T-wave example of FIG. 6B, a first relative maximum is identified with reference numeral 624, a first relative minimum is identified with reference numeral 626, and a second relative maximum is identified with reference numeral 628.

In the biphasic/negative example of FIG. 6C, a first relative minimum is identified with reference numeral 644, a first maximum is identified with reference numeral 646, a second minimum is identified with reference numeral 648, and a second maximum identified with reference numeral 650.

In the biphasic/positive example if FIG. 6D, a first relative maximum is identified with reference numeral 664, and first relative minimum is identified with reference numeral 666, a second relative maximum is identified with reference numeral 668, and a second relative minimum is identified with reference numeral 670.

The characteristic maximum and minimum points may be connected by straight line segments (dashed lines 610 and 612 in FIG. 6A, dashed lines 630 and 632 in FIG. 6B, dashed lines 652, 654, and 656 in FIG. 6C, and dashed lines 672, 674, and 676 in FIG. 6D) to represent the signal with primitive patterns. Based on these lines and characteristic points, rules may be established to classify any given T-wave morphology type based on the number and location of the characteristic points and the height and derivative of each line segment.

Referring again to FIG. 4, in step 410, a T-wave end search window is defined based upon the classification of the T-wave made in step 408. In examples, the T-wave end search window may be defined to include the last segment of the T-wave (e.g., line 612 in FIG. 6A, line 632 in FIG. 6B, line 656 in FIG. 6C and line 676 in FIG. 6D) identified in step 408. This results in, as examples, a T-wave end search window 614 in FIG. 6A, a T-wave end search window 634 in FIG. 6B, a T-wave end search window 658 in FIG. 6C, and a T-wave end search window 678 in FIG. 6D.

Next, in step 412 in method 400, the T-wave end is identified in the ECG beat by locating the maximum value of the arc-length curve for the ECG signal within the T-wave end search window identified in step 410. Examples of this are depicted in FIGS. 5A and 5B. In particular, in FIG. 5A, based upon the classification of the ECG signal 500, a T-wave end search window 516 is defined (in step 410 in FIG. 4). Following this, in step 412, a maximum value in arc-length curve 508 is identified, as represented by dashed line 518 in FIG. 5A.

Similarly, in FIG. 5B, based upon the classification of the ECG signal 520, a T-wave end search window 534 is defined (in step 410 in FIG. 4). Following this, in step 412, a maximum value in arc-length curve 528 is identified as the T-wave end, as represented by dashed line 536 in FIG. 5B.

Finally, in step 414 of method 400, a Q-T interval is computed using the onset point identified in step 404 and the T-wave end identified in step 412,

FIG. 7 is a flow diagram illustrating a method 700 for Q-T segment measurement according to one or more additional examples. A first step 702 in method 700 is the removal of baseline noise from each incoming ECG signal of each selected lead. In examples, the selection of at least one lead and the baseline removal may be performed according to the method depicted in FIGS. 3A and 3B herein.

In step 704 of method 700, an “average beat” for each selected ECG lead is computed for a predetermined averaging interval. In one example, an average beat for an ECG lead is computed over a 15-second averaging interval, although longer or shorter averaging intervals may be specified. In examples, an individual beat is included in creating an average beat over the 15-second averaging interval only if it is classified as normal or atrial-paced, and if it passes signal quality checks on the isoelectric point and Q-T segment regions and the remaining qualified beats meet the minimum threshold.

The signal quality check may be performed on the beat cycle waveform by traditional low-frequency and high-frequency noise measurement. The high frequency noise (“HFN”) of a lead may be simply measured by summing the second order difference of the beat cycle waveform with the QRS complex excluded. The low-frequency noise (“LFN” a lead may be simply measured by summing the 1-Hz low-passed beat cycle waveform.

The HFN, LFN, and the amplitude of the QRS complex are then compared to several thresholds, determining whether the beat passes the quality check. The amplitude of the QRS complex threshold is set as the minimum detection threshold, e.g., 0.2 mV There may be two different thresholds for HFN and LFN, one being fixed and another being determined from all beats in the previous beats. From the HFN and LFN of all beats in the averaging interval, the mean and standard deviation is computed, and multiple (e.g., ×2) of the mean is used as the dynamic threshold from the next averaging interval when the standard deviation is sufficiently low.

With continued reference to FIG. 7, in step 704, a beat will not be included in the averaging if it does not pass the quality check as described above. In some examples, the averaging is accumulated and updated upon each incoming new beat, such that each beat is only ⅛^{th} weighted in the average beat, and the stored accumulated average beat is ⅞^{th} weighted.

In block 706 of method 700, for each selected lead, a Q-T interval is computed for the average beat. The Q-T interval may be computed using the method 400 described above with reference to FIG. 4. This is referred to as an “interval complex measurement” for each lead.”

In block 708 of method 700, the Q-T segment is separately computed for each beat of each selected lead during the averaging interval. Again, the Q-T intervals may be computed using the method 400 described above with reference to FIG. 4.

In block 710, the Q-T segments of each beat from each selected lead, as computed in block 708, during the averaging interval are sorted, and upper and lower marginal measurements are discarded. In examples, the upper 25% and lower 25% of the computed Q-T segments are discarded.

In block 712, for each selected lead, the Q-T segments for each lead are averaged to produce, for each lead, an “each beat averaged measurement.” Then, in block 714, for each lead, an average of the interval complex measurement computed in block 706 and the each eat averaged measurement is computed. These results may then be reported to the clinician, in block 342 from the flow diagram of FIG. 3B, at the end of each averaging interval.

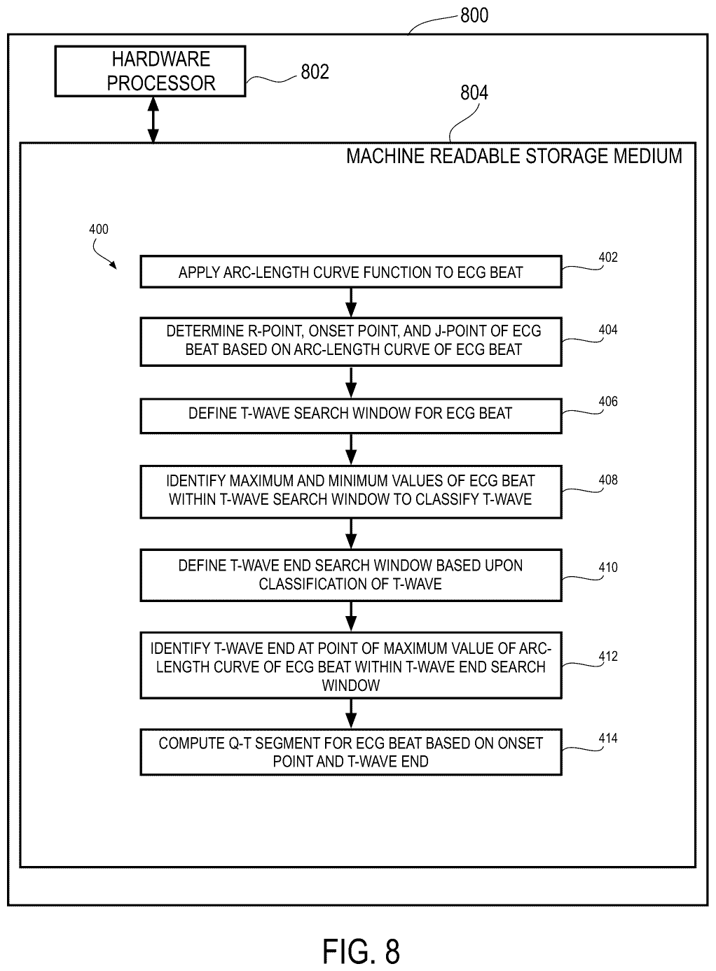

FIG. 8 is a block diagram representing a computing resource 800 implementing a method of ECG signal processing according to one or more examples. The computing resource 800 may be, for example and without limitation, a personal desktop computer (e.g., a personal computer), a mobile computing platform (e.g., a laptop or tablet), or a desktop computer accessing cloud computing resources. Computing resource 800 may include at least one hardware processor 802 and a non-transitory machine-readable storage medium 804. As illustrated, machine readable medium 804 may store instructions, that when executed by hardware processor 802 (either directly or via emulation/virtualization), cause hardware processor 802 to perform the method 400 of ECG signal processing described above with reference to FIG. 4.

In various examples, hardware processor 602 may be, for example and without limitation, a microcontroller, a central processing unit (“CPU”), a digital signal processor (“DSP”), a programmed logic array (“PLA”), or a custom processing circuit. Instructions may be executed by one or more processors, such as one or more central processing units (“CPU”), digital signal processors (“DSPs)”, general purpose microprocessors, application specific integrated circuits (“ASICs”), field programmable logic arrays (“FPGAs”), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A computer-readable media may be any available media that may be accessed by a computer. By way of example, such computer-readable media may comprise random access memory (“RAM”), read-only memory (“ROM”), electrically-erasable/programmable read-only memory (“EEPROM”), compact disc ROM (“CD-ROM”) or other optical disk storage, magnetic disk storage or other magnetic storage devices, or any other medium that may be used to carry or store desired program code in the form of instructions or data structures and that may be accessed by a computer. Disk and disc, as used herein, includes compact disc (“CD”), laser disc, optical disc, digital versatile disc (“DVD”), floppy disk and Blu-ray® disc where disks usually reproduce data magnetically, while discs reproduce data optically with lasers.

Note also that the software implemented aspects of the subject matter hereof are usually encoded on some form of program storage medium or implemented over some type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

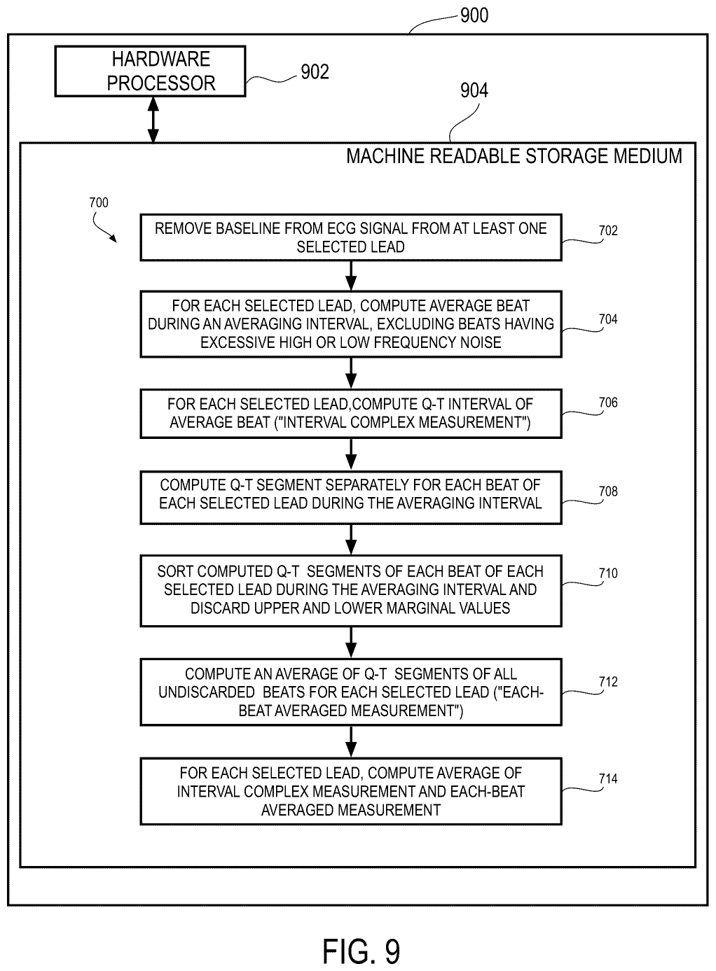

FIG. 9 is a block diagram representing a computing resource 900 implementing a method of ECG signal processing according to one or more examples. The computing resource 900 may be, for example and without limitation, a personal desktop computer (e.g., a personal computer), a mobile computing platform (e.g., a laptop or tablet), or a desktop computer accessing cloud computing resources. Computing resource 900 may include at least one hardware processor 902 and a non-transitory machine-readable storage medium 904. As illustrated, machine readable medium 904 may store instructions, that when executed by hardware processor 902 (either directly or via emulation/virtualization), cause hardware processor 902 to perform the method 900 of ECG signal processing described above with reference to FIG. 7.

Accordingly, a first embodiment comprises a method of processing of an electrocardiogram (“ECG”) signal from an ECG lead connected to a patient. The method comprises applying an arc-length curve function to an ECG beat of the ECG signal to determine an R-point, an onset point, and a J-point of the ECG beat. Next, a T-wave search window for the ECG beat, and maximum and minimum values of the ECG beat within the T-wave search window are identified.

A T-wave end search window is then defined based upon a classification of the T-wave, and T-wave end is identified at a point of maximum value of the arc-length curve of the ECG beat within the T-wave end search window. A Q-T segment value for the ECG beat is the computed based on the determined onset point and identified T-wave end.

Further in the method of the first embodiment, the arc-length curve function comprises:

$$ArcL [ i ] = {\sum }_{k = i - w}^{i} \sqrt{C + {( Δ y )}_{k}^{2}} \\ Δ {y}_{k} = ecgwaveform [ k ] - ecgwaveform [ k - 1 ]$$

where i is a time index, C is a constant related to sample interval in mSec, w is time window approximately equal to the width of the widest QRS complex during the predetermined period of time.

Further in the method of the first embodiment, determining the J-point comprises searching forward on the arc-length curve from the R-point to a first point with maximum value to be defined as the J-point.

Further in the method of the first embodiment, determining the onset point comprises searching backward on the arc-length curve from the J-point to a first point with minimum value to be defined as the onset point.

Further in the method of the first embodiment, computing an average beat from a plurality of beats occurring during a predetermined averaging interval.

From the average beat, an R-point, an onset point and a J-point of the average beat are determined, and an interval complex measurement reflecting a Q-T segment of the average beat based on the determined onset point and J-point is computed.

Further in the method of the first embodiment, a Q-T segment is computed separately for each beat during the averaging interval using the onset point and J-point of the average beat. The Q-T segments of each beat are sorter and upper and lower marginal values are discarded.

Then, an each beat averaged measurement comprising an average of undiscarded Q-T segments is computed, and an average of the interval complex measurement and the each beat averaged measurement is computed. The computed average of the interval complex measurement and the each beat averaged measurement may then be reported to a clinician.

At least one lead is selected to provide an ECG signal from the patient; and for each of the selected leads, an average beat from a plurality of beats occurring during a predetermined averaging interval is computed. The R-point, onset point, and the J-point of the average beat for each selected lead are computed, as well as an interval complex measurement reflecting a Q-T segment of the average beat based on the determined R-point, onset point and J-point;

Next, a Q-T segment is computed separately for each beat on each lead during the averaging interval using the onset point and J-point of the average beat. These Q-T segments are sorted, and upper and lower marginal values are discarded. The undiscarded Q-T segments are averaged to compute an each beat averaged measurement. The each beat averaged measurement is averaged with the interval complex measurement, and the result is presented to a clinician.

Further in the method of the first embodiment, the baseline noise of the ECG signal on each selected lead is removed prior to further processing.

Further, in the method of the first embodiment, the averaging interval may be 15 seconds, although longer or shorter averaging intervals may be used.

Further, in the method of the first embodiment, determining the onset point and J-point of the average beat comprises generating the arc-length curve function for the average beat.

Further, in the method of the first embodiment, computing the average beat from a plurality of beats occurring during a predetermined averaging interval comprises updating the average beat on each incoming new beat such that each beat contributes a fractional weighting to the average beat. Still further, low-frequency and high-frequency noise measurements may be performed on the ECG signal on each selected lead and beats with low or high frequency noise exceeding a predetermined threshold are excluded from the computation of the average beat.

Further, in the method of the first embodiment, determining an endpoint of a T-wave comprises searching within a T-wave end search window to identify a maximum value of the arc-length curve.

In a second embodiment, a physiological monitoring system for processing electrocardiogram (“ECG”) signals from a plurality of ECG leads connected to a patient, includes at least one lead for providing an ECG signal from the patient. Signal processing circuitry coupled to each of the selected leads, and may be programmed to compute an average beat from a plurality of beats occurring during a predetermined averaging interval.

An onset point and a J-point of the average beat are computed and used to compute an interval complex measurement reflecting a Q-T segment of the average beat. Then, a Q-T segment is computed separately for each beat during the averaging interval using the onset point and the J-point of the average beat/These Q-T segments are sorted, and Q-T segments at upper and lower marginal values are discarded. An each beat averaged measurement comprising an average of undiscarded Q-T segments is computed, and the each beat averaged measurement is averaged with the interval complex measurement. The result may then be reported to a clinician.

Further, in the system of the second embodiment, the signal processing circuitry may remove baseline noise from the ECG signals from the at least one selected lead. The averaging interval may be 15 seconds, although longer or shorter average intervals may be used.

Further, in the system of the second embodiment, the onset point and J-point of the average beat are determined by generating an arc-length curve function for the average beats. The arc-length curve function may comprise:

$$ArcL [ i ] = {\sum }_{k = i - w}^{i} \sqrt{C + {( Δ y )}_{k}^{2}} \\ Δ {y}_{k} = ecgwaveform [ k ] - ecgwaveform [ k - 1 ]$$

where i is a time index, C is a constant related to sample interval in mSec. w is time window approximately equal to the width of the widest QRS complex during the predetermined period of time.

Further, in the system of the second embodiment determining the J-point comprises searching forward on the arc-length curve from the R-point to a first point with maximum value to be defined as the J-point.

Further, in the system of the second embodiment, determining the onset point comprises searching backward on the arc-length curve from the J-point to a first point with minimum value to be defined as the onset point.

Further in the system of the second embodiment, the signal processing circuitry computes an average beat from a plurality of beats occurring during a predetermined averaging interval by updating the average beat on each incoming new beat such that each beat contributes a fractional weighting to the average beat.

Further in the system of the second embodiment, the signal processing circuitry performs low-frequency and high-frequency noise measurements of the ECG signal on each selected lead and excludes beats with low or high frequency noise exceeding a predetermined threshold from the computed average beat.

In a third embodiment, a computer-readable medium tangibly embodies instructions that, when executed by a processor, performs a method according to the first embodiment.

The detailed description is made with reference to the accompanying drawings and is provided to assist in a comprehensive understanding of various example embodiments of the present disclosure. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments. In addition, descriptions of well-known functions and constructions may be omitted for clarity and conciseness. Accordingly, those of ordinary skill in the art will recognize that various changes and modifications of the examples described herein can be made without departing from the spirit and scope of the present disclosure.

Use of the phrases “capable of,” “capable to,” “operable to,” “configured to,” or “programmed to” in one or more embodiments, refers to some apparatus, logic, hardware, and/or element designed in such a way to enable the use of the apparatus, logic, hardware, and/or element in a specified manner. Use of the phrase “exceed” in one or more embodiments, indicates that a measured value could be higher than a pre-determined threshold (e.g., an upper threshold), or lower than a pre-determined threshold (e.g., a lower threshold). When a pre-determined threshold range (defined by an upper threshold and a lower threshold) is used, the use of the phrase “exceed” in one or more embodiments could also indicate a measured value is outside the pre-determined threshold range (e.g., higher than the upper threshold or lower than the lower threshold). The subject matter of the present disclosure is provided as examples of apparatus, systems, methods, circuits, and programs for performing the features described in the present disclosure. However, further features or variations are contemplated in addition to the features described above. It is contemplated that the implementation of the components and functions of the present disclosure can be done with any newly arising technology that may replace any of the above-implemented technologies.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

What is claimed is:

1. A method of processing of an electrocardiogram (“ECG”) signal from an ECG lead connected to a patient, comprising:
applying an arc-length curve function to an ECG beat of the ECG signal;
determining an R-point, an onset point, and a J-point of the ECG beat based on the arc-length curve of the ECG beat;
defining a T-wave search window for the ECG beat;
identifying maximum and minimum values of the ECG beat within the T-wave search window;
defining a T-wave end search window based on a classification of the T-wave;
identifying a T-wave end at a point of maximum value of the arc-length curve of the ECG beat within the T-wave end search window;
computing a Q-T segment value for the ECG beat based on the determined onset point and identified T-wave end.

2. The method of claim 1, wherein the arc-length curve function comprises:
$$ArcL [ i ] = {\sum }_{k = i - w}^{i} \sqrt{C + {( Δ y )}_{k}^{2}} \\ Δ {y}_{k} = ecgwaveform [ k ] - ecgwaveform [ k - 1 ]$$
where i is a time index, C is a constant related to sample interval in mSec, w is time window approximately equal to the width of the widest QRS complex during the predetermined period of time.

3. The method of claim 2, wherein determining the J-point comprises searching forward on the arc-length curve from the R-point to a first point with maximum value to be defined as the J-point.

4. The method of claim 3, wherein determining the onset point comprises searching backward on the arc-length curve from the J-point to a first point with minimum value to be defined as the onset point.

5. The method of claim 1, wherein:
the ECG beat comprises an average beat from computed from a plurality of beats occurring during a predetermined averaging interval;
the method further comprising determining an R-point, an onset point and a J-point of the average beat;
computing an interval complex measurement reflecting a Q-T segment of the average beat based on the determined onset point and J-point;
computing a Q-T segment separately for each beat during the averaging interval using the onset point and J-point of the average beat;
sorting the computed Q-T segments of each beat and discarding upper and lower marginal values;
computing an each beat averaged measurement comprising an average of undiscarded Q-T segments;
computing an average of the interval complex measurement and the each beat averaged measurement; and
reporting the computed average of the interval complex measurement and the each beat averaged measurement to a clinician.

6. The method of claim 5, further comprising removing baseline noise from each of the ECG signal.

7. The method of claim 5, wherein the averaging interval is 15 seconds.

8. The method of claim 5, wherein computing an average beat from a plurality of beats occurring during a predetermined averaging interval comprises updating the average beat on each incoming new beat such that each beat contributes a fractional weighting to the average beat.

9. The method of claim 5, further comprising performing low-frequency and high-frequency noise measurements of the ECG signal on each selected lead and excluding beats with low or high frequency noise exceeding a predetermined threshold.

10. A physiological monitoring system for processing electrocardiogram (“ECG”) signals from a plurality of ECG leads connected to a patient, comprising:
at least one lead for providing an ECG signal from the patient;
signal processing circuitry coupled to each of the at least one selected leads, the signal processing circuitry programmed to:
compute an average beat from a plurality of beats occurring during a predetermined averaging interval;
determine an R-point, an onset point, and a J-point of the average beat;
compute an interval complex measurement reflecting a Q-T segment of the average beat based on the determined onset point and J-point;
compute a Q-T segment separately for each beat during the averaging interval using the R-point, onset point, and the J-point of the average beat;
sort the computed Q-T segments of each beat and discarding upper and lower marginal values;
compute an each beat averaged measurement comprising an average of undiscarded Q-T segments;
compute an average of the interval complex measurement and the each beat averaged measurement; and
report the computed average of the interval complex measurement and the each beat averaged measurement to a clinician.

11. The system of claim 10, wherein the signal processing circuitry removes baseline noise from the ECG signals from the at least one selected lead.

12. The system of claim 10, wherein the averaging interval is 15 seconds.

13. The system of claim 10, wherein the signal processing circuitry determines the onset point and J-point of the average beat by generating an arc-length curve function for the average beats.

14. The system of claim 13, wherein the arc-length curve function comprises:
$$ArcL [ i ] = {\sum }_{k = i - w}^{i} \sqrt{C + {( Δ y )}_{k}^{2}} \\ Δ {y}_{k} = ecgwaveform [ k ] - ecgwaveform [ k - 1 ]$$
where i is a time index, C is a constant related to sample interval in mSec. w is time window approximately equal to the width of the widest QRS complex during the predetermined period of time.

15. The system of claim 14, wherein determining the J-point comprises searching forward on the arc-length curve from the R-point to a first point with maximum value to be defined as the J-point.

16. The system of claim 15, wherein determining the onset point comprises searching backward on the arc-length curve from the J-point to a first point with minimum value to be defined as the onset point.

17. The system of claim 16, wherein determining an endpoint of a T-wave comprises searching forward on the arc-length curve from the J-point to a first point with maximum value to be defined as the end of the T-wave.

18. The system of claim 10, wherein the signal processing circuitry computes an average beat from a plurality of beats occurring during a predetermined averaging interval by updating the average beat on each incoming new beat such that each beat contributes a fractional weighting to the average beat.

19. The system of claim 10, wherein the signal processing circuitry performs low-frequency and high-frequency noise measurements of the ECG signal on each selected lead and excludes beats with low or high frequency noise exceeding a predetermined threshold from the computed average beat.

20. A computer-readable medium tangibly embodying instructions that, when executed by a processor, performs a method of processing of electrocardiogram (“ECG”) signals from a plurality of ECG leads connected to a patient, comprising:
selecting at least one lead to provide an ECG signal from the patient; and for each of the at least one selected leads:
computing an average beat from a plurality of beats occurring during a predetermined averaging interval;
determining an R-point, an onset point and a J-point of the average beat;
computing an interval complex measurement reflecting a Q-T segment of the average beat based on the determined onset point and J-point;
computing a Q-T segment separately for each beat during the averaging interval using the onset point and J-point of the average beat;
sorting the computed Q-T segments of each beat and discarding upper and lower marginal values;
computing an each beat averaged measurement comprising an average of undiscarded Q-T segments;
computing an average of the interval complex measurement and the each beat averaged measurement; and
reporting the computed average of the interval complex measurement and the each beat averaged measurement to a clinician.
